(19) United States
(12) Patent Application Publication
(10) Pub. No.: US 2023/0030597 A1
(43) Pub. Date: Feb. 2, 2023

(54) METHODS OF TREATING CANCER BY THE USE OF PD-1 AXIS INHIBITORS AND ANTI-PERIOSTIN ANTIBODIES

(71) Applicant: Boehringer Ingelheim IO Canada Inc., (US)

(72) Inventors: Arif JETHA, Toronto (CA); Johan FRANSSON, San Diego, CA (US); AJ Robert MCGRAY, Burlington (CA); Joanne HULME, Toronto (CA)

(21) Appl. No.: 17/641,825

(22) PCT Filed: Aug. 27, 2020
(86) PCT No.: PCT/CA2020/051164

§ 371 Date: Mar. 10, 2022

Related U.S. Application Data
(60) Provisional application No. 62/899,066, filed on Sep. 11, 2019.

(51) Int. Cl. C07K 16/18; A61P 35/00; C07K 16/28; A61P 13/10; A61P 1/00

(52) U.S. Cl. CPC C07K 16/18; A61P 35/00; C07K 16/2818; A61P 13/10; A61P 1/00; C07K 2317/565; A61K 2039/507

(57) ABSTRACT

Described herein are methods of treating cancer comprising administering to the individual (a) a PD-1 axis inhibitor; and (b) an inhibitor of periostin.

### SEQUENCE LISTING

The instant application contains a Sequence Listing which has been submitted electronically in ASCII format and is hereby incorporated by reference in its entirety. Said ASCII copy, created on Aug. 11, 2020, is named 01-3435-WO-1_SL.txt and is 19,155 bytes in size.

### BACKGROUND

Periostin is a matricellular protein that has been hypothesized to regulate a variety of physiological processes including epithelial-mesenchymal transition, cell-matrix interactions and inflammation. Its expression has been shown to be dysregulated in several pathologies including cancer and fibrosis, where overexpression of periostin is correlated with negative outcome. In addition to regulating extracellular remodeling by binding to other matricellular proteins such as fibronectin and collagen, periostin mediated integrin signaling has been shown to be critical for both migration of cancer cells and recruitment of immune cells in tumorigenic settings.

### SUMMARY

Described herein are methods, uses, and combinations of PD-1 axis inhibitors and periostin-inhibitors for the treatment of cancer. The methods described herein decrease the collagen content of tumors, reduce infiltration of suppressive myeloid cell populations, such as granulocytic cells and tumor associated macrophages while increasing macrophage polarization to an M1 phenotype, and increase the anti-tumor properties of tumor infiltrating T cells.

In one aspect, described herein, is a method of treating an individual afflicted with a cancer, the method comprising administering to the individual (a) a PD-1 axis inhibitor; and (b) an inhibitor of periostin. In certain embodiments, the inhibitor of periostin comprises an antibody or antigen binding fragment thereof that binds periostin. In certain embodiments, the inhibitor of periostin comprises an antibody or antigen binding fragment thereof that binds periostin, wherein the antibody or antigen binding fragment that binds periostin thereof comprises: (a) an immunoglobulin heavy chain CDR1 (CDR-H1) comprising the amino acid sequence set forth in SEQ ID NO: 1 (GYTFTSYG); (b) an immunoglobulin heavy chain CDR2 (CDR-H2) comprising an amino acid sequence set forth in any one of SEQ ID NOs: 2 (ISAYNGNT), 3 (ISAYSGNT), 4 (ISAYQGNT), 5 (ISAYTGNT), or 6 (ISAYDGNT); (c) an immunoglobulin heavy chain CDR3 (CDR-H3) comprising an amino acid sequence set forth in any one of SEQ ID NOs: 7 (DILVVPFDY), 8 (DVLVVPFDY), or 9 (DMLVVPFDY); (d) an immunoglobulin light chain CDR1 (CDR-L1) comprising the amino acid sequence set forth in SEQ ID NO: 10 (SSDIGSNR); (e) an immunoglobulin light chain CDR2 (CDR-L2) amino comprising the amino acid sequence set forth in SEQ ID NO: 11 (SND); and (f) an immunoglobulin light chain CDR3 (CDR-L3) comprising the amino acid sequence set forth in SEQ ID NO: 12 (AAWDDSLSTYV). In certain embodiments, the recombinant antibody or antigen binding fragment thereof that binds periostin is chimeric or humanized. In certain embodiments, the recombinant antibody or antigen binding fragment thereof that binds periostin is an IgG antibody. In certain embodiments, the recombinant antibody or antigen binding fragment thereof that binds periostin is a Fab, F(ab)₂, a single-domain antibody, or a single chain variable fragment (scFv). In certain embodiments, the recombinant antibody or antigen binding fragment thereof that binds periostin comprises an immunoglobulin heavy chain and an immunoglobulin light chain: (a) wherein the immunoglobulin heavy chain comprises an amino acid sequence at least about 90%, 95%, 97%, 99%, or 100% identical to that set forth in SEQ ID NO: 13; and (b) wherein the immunoglobulin light chain comprises an amino acid sequence at least about 90%, 95%, 97%, 99%, or 100% identical to that set forth in SEQ ID NO: 14, wherein asparagine number 55 of SEQ ID NO: 13 is asparagine, serine, glutamine, threonine, or aspartic acid, and wherein methionine number 100 of SEQ ID NO: 13 is methionine, isoleucine, or valine. In certain embodiments, the antibody or antigen binding fragment thereof that binds periostin has an IC50 of less than about 50 nanomolar in a cell adhesion assay performed with human lung fibroblast cells and/or mouse fibroblast cells. In certain embodiments, the PD-1 axis inhibitor is an inhibitor of PD-1, PDL-1, or PDL-2 signaling is an antibody or fragment thereof that binds to PD-1. In certain embodiments, the PD-1 axis inhibitor is an antibody or fragment thereof that binds to PD-1.

A PD-1 pathway inhibitor within the meaning of this invention and all of its embodiments is a compound that inhibits the interaction of PD-1 with its receptor(s). A PD-1 pathway inhibitor is capable to impair the PD-1 pathway signaling, preferably mediated by the PD-1 receptor. The PD-1 inhibitor may be any inhibitor directed against any member of the PD-1 pathway capable of antagonizing PD-1 pathway signaling. The inhibitor may be an antagonistic antibody targeting any member of the PD-1 pathway, preferably directed against PD-1 receptor, PD-L1 or PD-L2. Also, the PD-1 pathway inhibitor may be a fragment of the PD-1 receptor or the PD-1 receptor blocking the activity of PD1 ligands.

PD-1 antagonists are well-known in the art, e.g. reviewed by Li et al., Int. J. Mol. Sci. 2016, 17, 1151 (incorporated herein by reference). Any PD-1 antagonist, especially antibodies, such as those disclosed by Li et al. as well as the further antibodies disclosed herein below, can be used according to the invention. Preferably, the PD-1 antagonist of this invention and all its embodiments is selected from the group consisting of the following antibodies: pembrolizumab (anti-PD-1 antibody); nivolumab (anti-PD-1 antibody); pidilizumab (anti-PD-1 antibody); tislelizumab (anti PD-1); spartalizumab (PDR-001) (anti-PD-1 antibody), preferably ezabenlimab (anti-PD-1 antibody).

In certain embodiments, the PD-1 axis inhibitor is an antibody that specifically binds PDL-1 or PDL-2. In certain non-limiting embodiments, the antibody that specifically binds PDL-1 or PDL-2 comprises durvalumab, atezolizumab, avelumab, AMP-224, MEDI0680 (AMP-514), BMS-936559 (MDX-1105), toripalimab (JS001-PD-1), cemiplimab (REGN2810), camrelizumab (SHR-1210), dostarlimab (TSR-042) cetrelimab (JNJ-63723283), or FAZ053, or a PDL-1 or PDL-2 binding fragment thereof. In certain embodiments, the inhibitor of PD-1, PDL-1, or PDL-2 signaling comprises an Fc-Fusion protein that binds PD-1, PDL-1, or PDL-2. In certain embodiments, the Fc-Fusion protein comprises AMP-224 or a PD-1 binding fragment thereof. In certain embodiments, the inhibitor of PD-1, PDL-1, or PDL-2 signaling comprises a small molecule inhibitor of PD-1, PDL-1, or PDL-2. In certain embodiments, the small molecule inhibitor of signaling through PD-1, PDL-1, or PDL-2 comprises on or more of: N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo[b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-α-glutamyl-L-seryl-L-phenylalanyl)-L-lysyl-L-phenylalanyl-L-arginyl-L-valyl-L-threonyl-L- glutaminyl-L-leucyl-L-alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L- tryptophyl-N-methyl-L-norleucyl-N-methyl-L-norleucyl-L-arginyl-L-cysteinyl-, cyclic (1→14)-thioether; or a derivative or analog thereof.

In certain embodiments, the individual has developed progressive disease after treatment with a checkpoint inhibitor as a monotherapy. In certain embodiments, the checkpoint inhibitor comprises a PD-1 access inhibitor. In certain embodiments, the PD-1 axis inhibitor and the inhibitor of periostin are administered separately. In certain embodiments, the PD-1 axis inhibitor and the inhibitor of periostin are administered on the same day. In certain embodiments, the PD-1 axis inhibitor and the inhibitor of periostin are administered on different days. In certain embodiments, the cancer comprises glioblastoma, pancreatic cancer, breast cancer, bladder cancer, kidney cancer, head and neck cancer, ovarian cancer, colon cancer, cervical cancer, prostate cancer, or lung cancer.

In another aspect, described herein, is an antibody or antigen binding fragment thereof that binds periostin for use in a patient also being treated with a PD-1 axis inhibitor. In certain embodiments, the antibody or antigen binding fragment thereof that binds periostin comprises: (a) an immunoglobulin heavy chain CDR1 (CDR-H1) comprising the amino acid sequence set forth in SEQ ID NO: 1 (GYTFTSYG); (b) an immunoglobulin heavy chain CDR2 (CDR-H2) comprising an amino acid sequence set forth in any one of SEQ ID NOs: 2 (ISAYNGNT), 3 (ISAYSGNT), 4 (ISAYQGNT), 5 (ISAYTGNT), or 6 (ISAYDGNT); (c) an immunoglobulin heavy chain CDR3 (CDR-H3) comprising an amino acid sequence set forth in any one of SEQ ID NOs: 7 (DILVVPFDY), 8 (DVLVVPFDY), or 9 (DMLVVPFDY); (d) an immunoglobulin light chain CDR1 (CDR-L1) comprising the amino acid sequence set forth in SEQ ID NO: 10 (SSDIGSNR); (e) an immunoglobulin light chain CDR2 (CDR-L2) amino comprising the amino acid sequence set forth in SEQ ID NO: 11 (SND); and (0 an immunoglobulin light chain CDR3 (CDR-L3) comprising the amino acid sequence set forth in SEQ ID NO: 12 (AAWDDSLSTYV).

In certain embodiments, the recombinant antibody or antigen binding fragment thereof that binds periostin is chimeric or humanized. In certain embodiments, the recombinant antibody or antigen binding fragment thereof that binds periostin is an IgG antibody. In certain embodiments, the recombinant antibody or antigen binding fragment thereof that binds periostin is a Fab, F(ab)₂, a single-domain antibody, or a single chain variable fragment (scFv). In certain embodiments, the recombinant antibody or antigen binding fragment thereof that binds periostin comprises an immunoglobulin heavy chain and an immunoglobulin light chain: (a) wherein the immunoglobulin heavy chain comprises an amino acid sequence at least about 90%, 95%, 97%, 99%, or 100% identical to that set forth in 13; and (b) wherein the immunoglobulin light chain comprises an amino acid sequence at least about 90%, 95%, 97%, 99%, or 100% identical to that set forth in SEQ ID NO: 14, wherein asparagine number 55 of SEQ ID NO: 13 is asparagine, serine, glutamine, threonine, or aspartic acid, and wherein methionine number 100 of SEQ ID NO: 13 is methionine, isoleucine, or valine. In certain embodiments, the antibody or antigen binding fragment thereof that binds periostin has an IC50 of less than about 50 nanomolar in a cell adhesion assay performed with human lung fibroblast cells and/or mouse fibroblast cells. In certain embodiments, the PD-1 axis inhibitor is an inhibitor of PD-1, PDL-1, or PDL-2 signaling is an antibody or fragment thereof that binds to PD-1. In certain embodiments, the PD-1 axis inhibitor is an antibody or fragment thereof that binds to PD-1. In certain embodiments, the antibody or fragment thereof that binds to PD-1 comprises Pembrolizumab, Nivolumab, AMP-514, Tislelizumab, Spartalizumab, preferably ezabenlimab, or a PD-1 binding fragment thereof. In certain embodiments, the PD-1 axis inhibitor is an antibody that specifically binds PDL-1 or PDL-2. In certain embodiments, the antibody that specifically binds PDL-1 or PDL-2 comprises durvalumab (MEDI4736), atezolizumab (MPDL3280A), avelumab (MSB0010718C), BMS-936559 (MDX-1105), AMP-224, MEDI0680 (AMP-514), cemiplimab (REGN2810), toripalimab (JS001-PD-1), camrelizumab (SHR-1210), dostarlimab (TSR-042), cetrelimab (JNJ-63723283), or FAZ053, or a PDL-1 or PDL-2 binding fragment thereof. In certain embodiments, the inhibitor of PD-1, PDL-1, or PDL-2 signaling comprises an Fc-Fusion protein that binds PD-1, PDL-1, or PDL-2. In certain embodiments, the Fc-Fusion protein comprises AMP-224 or a PD-1 binding fragment thereof. In certain embodiments, the inhibitor of PD-1, PDL-1, or PDL-2 signaling comprises a small molecule inhibitor of PD-1, PDL-1, or PDL-2. In certain embodiments, the small molecule inhibitor of signaling through PD-1, PDL-1, or PDL-2 comprises on or more of: N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo[b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-α-glutamyl-L-seryl-L-phenylalanyl)-L-lysyl-L-phenylalanyl-L-arginyl-L-valyl-L-threonyl-L- glutaminyl-L-leucyl-L-alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L- tryptophyl-N-methyl-L-norleucyl-N-methyl-L-norleucyl-L-arginyl-L-cysteinyl-, cyclic (1→14)-thioether; or a derivative or analog thereof. In certain embodiments, the individual is afflicted with cancer. In certain embodiments, the cancer comprises glioblastoma, pancreatic cancer, breast cancer, bladder cancer, kidney cancer, head and neck cancer, ovarian cancer, colon cancer, cervical cancer, prostate cancer, or lung cancer.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 illustrates inhibition of periostin mediated cell attachment by 78 sequence unique IgGs tested at a single concentration of 500 nM.

FIG. 2 illustrates tumor growth in the mouse MB49 bladder cancer model following treatment with NB0828 or vehicle control.

FIG. 3 illustrates impact of NB0828 treatment on accumulation of intratumoral myeloid cells. MB49 tumor-bearing mice were treated with NB0828 or vehicle as described in FIG. 2. Data is presented as percent of total CD45+ immune infiltrate.

FIG. 4 illustrates changes in total tumor collagen content following treatment with NB0828. MB49 tumor-bearing mice were treated as described in FIG. 2 and total tumor collagen content of endpoint MB49 tumors was assessed as described in the methods.

FIG. 5 illustrates tumor growth in the mouse CT26 colon cancer model following treatment with NB0828 or vehicle control.

FIG. 6 illustrates reduced intratumoral accumulation of granulocytic cells/TAMs (Tumor associated macrophages) and macrophage skewing towards an M1 phenotype in NB0828 treated CT26 tumor-bearing mice.

FIG. 7 illustrates increased accumulation of CD8+ and CD4+ tumor infiltrating lymphocytes (TILs) and enhanced CD8+ TIL function in NB0828 treated CT26 tumor-bearing mice.

FIG. 8 illustrates tumor growth in the mouse MC38 colon cancer model following treatment with NB0828 or vehicle control.

FIGS. 9A-9D illustrate that in the MC38 colon cancer model NB0828 decreases the overall amount of tumor associated macrophages (9A), while increasing pro-inflammatory type I macrophages (9B), and CD8+ T cells (9C), and that tumor efficacy is dependent on C8+ T cells (9D).

FIGS. 10A-10C illustrates that the combination of PD-1 and NB0828 improves response to in the MC38 colon cancer model compared to PD-1 alone in controlling both with respect to tumor size (10A) and survival (10B); animals that survived the first challenge are protected from rechallenge (10C).

FIG. 11 illustrates that NB0828 overcomes resistance to anti-PD-1 in established MC38 tumors.

FIG. 12A-12D illustrates that in PD-1 resistant tumors NB0828 improves the frequency (12A) and function of CD8+ TILs (12B) while reducing total TAMs (12C) and promoting accumulation of immunostimulatory M1 TAMs (12D).

### DETAILED DESCRIPTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “combination” or “combination treatment” can refer either to concurrent administration of the articles to be combined or sequential administration of the articles to be combined. As described herein, when the combination refers to sequential administration of the articles, the articles can be administered in any temporal order. Articles can be administered separately on different days or the same day each article according to a schedule that maximizes bioavailability, reduces side effects, maximizes therapeutic potential, or any combination thereof.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein the term an “effective amount” refers to the amount of a therapeutic that causes a biological effect when administered to a mammal. Biological effects include, but are not limited to, inhibition or blockade a receptor ligand interaction, reduction in enzymatic activity of the target, reduced tumor growth, reduced tumor metastasis, increased infiltration of CD8+ T cells to tumor sites, reduced total macrophages in tumor sites, increased infiltration of M1 macrophages, increases in the M1/M2 ratio or prolonged survival of an animal bearing a tumor. A “therapeutic amount” is the concertation of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

Among the provided antibodies are monoclonal antibodies, polyclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J Immunol. 150:880-887 (1993); Clarkson et al., Nature 352:624-628 (1991)). Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments, the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn297 (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63(1):78-85). However, Asn₂₉₇ may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and 5400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising one or more nucleic acids encoding an antibody described herein integrated at a genomic location; and (b) a cryoprotectant. In certain embodiments, the cryoprotectant comprises glycerol. In certain embodiments, the master cell bank comprises: (a) a CHO cell line comprising a nucleic acid encoding an antibody with (i) a heavy chain amino acid sequence at least 90% identical to that set forth by SEQ ID NO: 13; and (ii) a light chain amino acid sequence at least 90% identical to that set forth by SEQ ID NO: 14 integrated at a genomic location; and (b) a cryoprotectant. In certain embodiments, the cryoprotectant comprises glycerol. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, dialysis, desalting, protein A, protein G, protein A/G, or protein L purification, and/or ion exchange chromatography.

### Anti-Periostin Antibodies

Described herein are antibodies that block periostin function. Such antibodies are useful for the treatment of cancer. The antibodies described herein decrease the collagen content of tumors, reduce infiltration of granulocytes and tumor associated macrophages while increasing macrophage polarization to an M1 phenotype, and increase the accumulation and anti-tumor properties of tumor infiltrating T cells. In certain embodiments, the anti-periostin antibodies decrease tumor collagen content by at least about 5%, 10%, 15%, 20%, 25%, 30%, 35%, or 40% compared to an untreated or control treated individual. In certain embodiments, the anti-periostin antibodies reduce infiltration of granulocytes and tumor associated macrophages by at least about 20%, 25%, 30%, 35%, 40%, 45%, or 50% compared to an untreated or control treated individual. In certain embodiments, the anti-periostin antibodies reduce infiltration of CD11b+ cells by at least about 20%, 25%, 30%, 35%, 40%, 45%, or 50% compared to an untreated or control treated individual. In certain embodiments, the anti-periostin antibodies increase polarization of tumor associated macrophages to the M1 type (CD11b+, MHC class II+, CD206−) by at least about 20%, 25%, 30%, 35%, 40%, 45%, or 50% compared to an untreated or control treated individual. In certain embodiments, the anti-periostin antibodies increase accumulation of CD4+ and/or CD8+ T cells in a tumor by at least about 20%, 25%, 30%, 35%, 40%, 45%, or 50% compared to an untreated or control treated individual. In certain embodiments, the anti-periostin antibodies increase production of interferon gamma of tumor infiltrating CD8+ T cells by at least about 20%, 25%, 30%, 35%, 40%, 45%, or 50% compared to an untreated or control treated individual.

Described herein is a recombinant antibody or antigen binding fragment thereof that binds periostin, wherein the antibody or antigen binding fragment thereof comprises: (a) an immunoglobulin heavy chain CDR1 (CDR-H1) comprising the amino acid sequence set forth in SEQ ID NO: 1 (GYTFTSYG); (b) an immunoglobulin heavy chain CDR2 (CDR-H2) comprising an amino acid sequence set forth in any one of SEQ ID NOs: 2 (ISAYNGNT), 3 (ISAYSGNT), 4 (ISAYQGNT), 5 (ISAYTGNT), or 6 (ISAYDGNT); (c) an immunoglobulin heavy chain CDR3 (CDR-H3) comprising an amino acid sequence set forth in any one of SEQ ID NOs: 7 (DILVVPFDY), 8 (DVLVVPFDY), or 9 (DMLVVPFDY); (d) an immunoglobulin light chain CDR1 (CDR-L1) comprising the amino acid sequence set forth in SEQ ID NO: 10 (SSDIGSNR); (e) an immunoglobulin light chain CDR2 (CDR-L2) amino comprising the amino acid sequence set forth in SEQ ID NO: 11 (SND); (0 and an immunoglobulin light chain CDR3 (CDR-L3) comprising the amino acid sequence set forth in SEQ ID NO: 12 (AAWDDSLSTYV). In certain embodiments, the antibody is a humanized or chimeric antibody. In certain embodiments, the antibody is an IgG antibody. In certain embodiments, the antibodies described herein can comprise an Fc portion with a lack of effector function. In certain embodiments, the antibody has an IC50 of less than about 50 nanomolar in a cell adhesion assay performed with human lung fibroblast cells and/or mouse fibroblast cells. In certain embodiments, the antibody has an IC50 of less than about 40 nanomolar in a cell adhesion assay performed with human lung fibroblast cells and/or mouse fibroblast cells. In certain embodiments, the antibody has an IC50 of less than about 30 nanomolar in a cell adhesion assay performed with human lung fibroblast cells and/or mouse fibroblast cells.

Also described herein is a recombinant antibody or antigen binding fragment thereof that binds periostin, comprising an immunoglobulin heavy chain and an immunoglobulin light chain: (a) wherein the immunoglobulin heavy chain comprises an amino acid sequence at least about 90%, 95%, 97%, 99%, or 100% identical to that set forth in SEQ ID NO: 13; and (b) wherein the immunoglobulin light chain comprises an amino acid sequence at least about 90%, 95%, 97%, 99%, or 100% identical to that set forth in SEQ ID NO: 14, wherein asparagine number 55 of SEQ ID NO 13: is asparagine, serine, glutamine, threonine, or aspartic acid, and wherein methionine number 100 of SEQ ID NO: 13 is methionine, isoleucine, or valine. In certain embodiments, the antibody is an IgG antibody. In certain embodiments, the antibodies described herein can comprise an Fc portion with a lack of effector function. In certain embodiments, the antibody has an IC50 of less than about 50 nanomolar in a cell adhesion assay performed with human lung fibroblast cells and/or mouse fibroblast cells. In certain embodiments, the antibody has an IC50 of less than about 40 nanomolar in a cell adhesion assay performed with human lung fibroblast cells and/or mouse fibroblast cells. In certain embodiments, the antibody has an IC50 of less than about 30 nanomolar in a cell adhesion assay performed with human lung fibroblast cells and/or mouse fibroblast cells.

### PD-1 Axis Inhibitors

The PD-1 axis is the signaling pathway through which PD-1 exerts an inhibitory effect on T-cell responses and includes the PD-1 interaction with PDL-1 or PDL-2. The periostin-binding polypeptides and antibodies described herein can be combined with a PD-1 axis inhibitor and deployed in a method to treat a tumor, cancer or other neoplasm. In certain embodiments, the periostin binding polypeptides and antibodies described herein can be combined with a PD-1 axis inhibitor in a pharmaceutical composition useful for treating a cancer, tumor, or other neoplasm. The NB0828 antibody described herein can be combined with a PD-1 axis inhibitor and deployed in a method to treat a tumor, cancer or other neoplasm. In certain embodiments, the NB0828 antibody described herein can be combined with a PD-1 axis inhibitor in a pharmaceutical composition useful for treating a cancer, tumor, or other neoplasm.

The PD-1 axis inhibitor utilized in the compositions and methods herein can inhibit signaling through PD-1 (CD279), PDL-1 (CD274), or PDL-2 (CD273). The inhibitor can be an antibody or antibody fragment, a soluble ligand-Fc fusion construct, or a small molecule inhibitor. In certain embodiments, the PD-1 axis inhibitor comprises an antibody or PD-1 binding fragment thereof. In certain embodiments, the antibody or antigen binding fragment that specifically binds PD-1 (CD279) comprises pembrolizumab, nivolumab, AMP-514 (MEDI0680), spartalizumab, tislelizumab (BGB-A317), pidilizumab, preferably ezabenlimab (CAS #2249882-54-8) (anti-PD-1 antibodies), or a PD-1 (CD279) binding fragment thereof.

Specifically, the anti-PD-1 antibody molecule described herein is ezabenlimab comprising a heavy chain comprising the amino acid sequence of SEQ ID NO.:19 and a light chain comprising the amino acid sequence of SEQ ID NO.:20.

In certain embodiments, the PD-1 Axis inhibitor is a PD-L2 Fc fusion protein (e.g., AMP-224). In certain embodiments, the PD-1 axis inhibitor comprises an antibody or PDL-1 binding fragment that specifically binds PDL-1 (CD274). In certain embodiments, the antibody or antigen binding fragment that specifically binds to PDL-1 (CD274) comprises durvalumab (MEDI 4376), atezolizumab (MPDL3280A), avelumab (MSB0010718C), BMS-936559 (MDX-1105), MEDI0680 (AMP-514), cemiplimab (REGN2810), toripalimab (JS001-PD-1), camrelizumab (SHR-1210), dostarlimab (TSR-042), cetrelimab (JNJ-63723283), or FAZ053, or a PDL-1 (CD274) binding fragment thereof. In certain embodiments, the PD-1 axis inhibitor comprises an antibody or PDL-2 binding fragment thereof that specifically binds PDL-2 (CD273).

In certain embodiments, the PD-1 axis inhibitor comprises one or more a small molecule inhibitors such as N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo[b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-α-glutamyl-L-seryl-L-phenylalanyl)-L-lysyl-L-phenylalanyl-L-arginyl-L-valyl-L-threonyl-L-glutaminyl-L-leucyl-L-alanyl-L-prolyl-L-lysyl-L-alanyl-L-glutaminyl-L-isoleucyl-L-lysyl; (2S)-1- [[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L- tryptophyl-N-methyl-L-norleucyl-N-methyl-L-norleucyl-L-arginyl-L-cysteinyl-, cyclic (1→14)-thioether; or a derivative or analog thereof.

In certain embodiments, the PD-1 axis inhibitors can be administered by any route suitable for the administration of a small molecule polypeptide or antibody-containing pharmaceutical composition, such as, for example, subcutaneous, intraperitoneal, intravenous, intramuscular, intratumoral, intracerebral, or oral. In certain embodiments, PD-1 axis inhibiting antibodies are administered intravenously. In certain embodiments, the PD-1 axis inhibiting antibodies are administered on a suitable dosage schedule, for example, weekly, twice weekly, monthly, twice monthly, once every two weeks, once every three weeks, or once every four weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. In certain embodiments, the therapeutically acceptable amount is between about 5 mg/kg and about 15 mg/kg. In certain embodiments, the therapeutically acceptable amount is about 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. In one example, Durvalumab can be administered at a dosage of about 10 mg/kg once every two weeks.

In certain embodiments, administration to an individual of the PD-1 axis inhibitors can be at a flat dosage level of between about 100 milligrams and about 1000 milligrams. In certain embodiments, administration to an individual of the PD-1 axis inhibitors can be at flat dosage level of between about 200 milligrams and about 800 milligrams, between about 200 milligrams and about 600 milligrams, between about 200 milligrams and about 500 milligrams, between about 300 milligrams and about 500 milligrams. In certain embodiments, administration to an individual of the PD-1 axis inhibitors can be at a flat dosage level of about 100, 150, 200, 250, 300, 350, 400, 450, 500, 550, 600, 650, 700, 750, 800, 850, 900, 950 or 1000 milligrams. In certain embodiments, administration to an individual of the PD-1 axis inhibitors can be at level suitable for monotherapy. For example, Nivolumab can be administered at a dosage of about 240 milligrams every two weeks or about 480 milligrams every four weeks. In another example Pembrolizumab can be administered at about 200 milligrams once every three weeks.

### Therapeutic Methods

The antibodies disclosed herein are antibodies useful for the treatment of a cancer or tumor. Treatment refers to a method that seeks to improve or ameliorate the condition being treated. With respect to cancer treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that while an antibody may be safe and effective, not all individuals will respond equally to a treatment that is administered, nevertheless these individuals are considered to be treated.

The methods described herein also encompass methods of treating individuals with caner by administering a combination of PD-1 axis inhibitor and periostin binding antibody. In certain embodiments, the PD-1 axis inhibitor and periostin binding antibody can be administered separately. In certain embodiments, the PD-1 axis inhibitor and periostin binding antibody can be administered separately on the same day of treatment. In certain embodiments, the PD-1 axis inhibitor and periostin binding antibody can be administered separately each according to its own administration schedule. In certain embodiments, the separate administration schedules are designed to maximize the PK/PD characteristics of each inhibitor. In certain embodiments, the periostin binding antibody comprises NB0828 or an antibody with the CDRs of NB0828. In certain embodiments, the PD-1 axis inhibitor comprises ezabenlimab (CAS #2249882-54-8) or an antibody with the CDRs of ezabenlimab as disclosed herein.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, or liver tumors. In certain embodiments, tumors or cancers which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiform, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, or lung cancer.

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated.

### Dosage Schedules of Combination Therapies

A combination treatment comprising a periostin binding antibody or polypeptide and a PD-1 axis inhibitor can be administered in a variety of ways. The periostin-binding antibody or polypeptide and the PD-1 axis inhibitor can be administered at the same time on the same schedule, or at different times and on different schedules. When administered at the same time the administration can be by way of separate formulations or a single formulation comprising both the periostin-binding polypeptide and the PD-1 axis inhibitor. Modes of administration can be mixed, for example a periostin-binding polypeptide can be administered intravenously while a PD-1 axis inhibitor can be administered orally or by parenteral injection. In certain embodiments, a periostin-binding polypeptide is administered intravenously, parenterally, subcutaneously, intratumorally, or orally. In certain embodiments, a PD-1 axis inhibitor is administered intravenously, parenterally, subcutaneously, intratumorally, or orally.

When a combination treatment is administered to an individual on the same schedule the periostin-binding polypeptide and the PD-1 axis inhibitor can be administered once every week, once every two weeks, once every three weeks, or once every four weeks. The periostin-binding polypeptide and the PD-1 axis inhibitor can be administered separately or as a single formulation. NB0828 and a PD-1 axis inhibitor can be administered separately or as a single formulation.

When a combination treatment is administered to an individual on a different schedule the periostin-binding polypeptide and the PD-1 axis inhibitor can be alternated. In certain embodiments, a PD-1 axis inhibitor can be administered to an individual one or more times before administration of a periostin-biding polypeptide. A periostin-binding polypeptide can be administered within 1 day, 2 days, 3 days, 4 days, 5 days, or 6 days of administration of a PD-1 axis inhibitor. A periostin-binding polypeptide can be administered within 1 week, 2 weeks, 3 weeks, or 4 weeks of administration of a PD-1 axis inhibitor. The NB0828 antibody can be administered within 1 day, 2 days, 3 days, 4 days, 5 days, or 6 days of administration of a PD-1 axis inhibitor. The NB0828 antibody can be administered within 1 week, 2 weeks, 3 weeks, or 4 weeks of administration of a PD-1 axis inhibitor.

A periostin-biding polypeptide can be administered to an individual one or more times before administration of a PD-1 axis inhibitor. In certain embodiments, a PD-1 axis inhibitor can be administered within 1 day, 2 days, 3 days, 4 days, 5 days, or 6 days of administration of a periostin-binding polypeptide. In certain embodiments, a PD-1 axis inhibitor can be administered within 1 week, 2 weeks, 3 weeks, or 4 weeks of administration of a periostin-binding polypeptide. In certain embodiments, a PD-1 axis inhibitor can be administered within 1 day, 2 days, 3 days, 4 days, 5 days, or 6 days of administration of the NB0828 antibody. In certain embodiments, a PD-1 axis inhibitor can be administered within 1 week, 2 weeks, 3 weeks, or 4 weeks of administration of the NB0828 antibody.

In certain embodiments, a periostin binding polypeptide can be administered to an individual once every week and a PD1-axis inhibitor can be administered to an individual every week, every two weeks, every three weeks or every four weeks. In certain embodiments, a periostin binding polypeptide can be administered to an individual once every two weeks and a PD1-axis inhibitor can be administered to an individual every week, every two weeks, every three weeks or every four weeks. In certain embodiments, a periostin binding polypeptide can be administered to an individual once every three weeks and a PD1-axis inhibitor can be administered to an individual every week, every two weeks, every three weeks or every four weeks. In certain embodiments, a periostin binding polypeptide can be administered to an individual once every four weeks and a PD1-axis inhibitor can be administered to an individual every week, every two weeks, every three weeks or every four weeks. In certain embodiments, a PD1-axis inhibitor can be administered to an individual once every week and a periostin-binding polypeptide can be administered to an individual every week, every two weeks, every three weeks or every four weeks. In certain embodiments, a PD1-axis inhibitor e can be administered to an individual once every two weeks and a periostin-binding polypeptide can be administered to an individual every week, every two weeks, every three weeks or every four weeks. In certain embodiments, a PD1-axis inhibitor can be administered to an individual once every three weeks and a periostin-binding polypeptide can be administered to an individual every week, every two weeks, every three weeks or every four weeks. In certain embodiments, a PD1-axis inhibitor can be administered to an individual once every four weeks and a periostin-binding polypeptide can be administered to an individual every week, every two weeks, every three weeks or every four weeks. In certain embodiments, NB0828 can be administered to an individual one or more times before administration of a PD-1 axis inhibitor. In certain embodiments, NB0828 can be administered to an individual once every week and a PD1-axis inhibitor can be administered to an individual every week, every two weeks, every three weeks or every four weeks. In certain embodiments, NB0828 can be administered to an individual once every two weeks and a PD1-axis inhibitor can be administered to an individual every week, every two weeks, every three weeks or every four weeks. In certain embodiments, NB0828 can be administered to an individual once every three weeks and a PD1-axis inhibitor can be administered to an individual every week, every two weeks, every three weeks or every four weeks. In certain embodiments, NB0828 can be administered to an individual once every four weeks and a PD1-axis inhibitor can be administered to an individual every week, every two weeks, every three weeks or every four weeks.

A combination treatment according to the current disclosure may comprise combinations wherein one or both of the activate ingredients (e.g., a periostin-binding polypeptide and an inhibitor of PD-1) is not effective by itself, but is effective when administered as a part of a combination treatment. In certain embodiments, an inhibitor of PD-1 is administered at a level not effective for monotherapy, but effective in combination with a periostin-binding polypeptide. In certain embodiments, an inhibitor of PD-1 is administered at a level not effective for monotherapy, but effective in combination with the NB0828 antibody. In certain embodiments, a periostin-binding polypeptide is administered at a level not effective for monotherapy, but effective in combination with an inhibitor of PD-1. In certain embodiments, NB0828 is administered at a level not effective for monotherapy, but effective in combination with an inhibitor of PD-1. In certain embodiments, both a periostin-binding polypeptide, and an inhibitor of PD-1 is administered at a level not effective for monotherapy, but is effective in combination. In certain embodiments, both NB0828, and an inhibitor of PD-1 is administered at a level not effective for monotherapy, but is effective in combination.

### Pharmaceutically Acceptable Excipients, Carriers, and Diluents

In certain embodiments, the anti-periostin antibodies of the current disclosure are included in a pharmaceutical composition comprising one or more pharmaceutically acceptable excipients, carriers, and diluents. In certain embodiments, the antibodies of the current disclosure are administered suspended in a sterile solution. In certain embodiments, the solution comprises 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional component selected from: instructions for use; a diluent, an excipient, a carrier, and a device for administration.

In certain embodiments, described herein is a method of preparing a cancer treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### EXAMPLES

The following illustrative examples are representative of embodiments of compositions and methods described herein and are not meant to be limiting in any way.

### Example 1—Antibody Generation and Screening

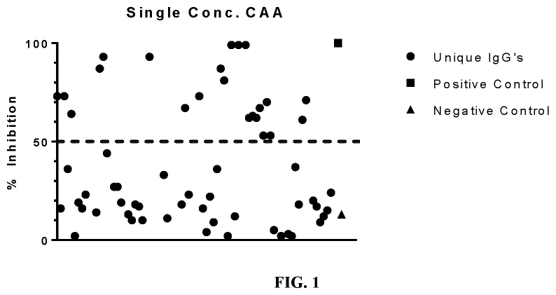

A phage display antibody discovery campaign was performed to isolate binders against periostin using a fully human phage library. Briefly, three rounds of panning were conducted using either recombinant human periostin, recombinant mouse periostin, or combinations thereof, with an emphasis on identifying mouse cross-reactive binders. From this panning strategy, 78 sequence unique ScFv's that cross-react to mouse periostin were identified and produced in a human IgG1 format for functional screening in a cell attachment assay. See FIG. 1.

Recombinant human or mouse periostin was coated on 96 well plates overnight at 4° C. The next day, plates were washed with PBS and blocked with 2% BSA for 1 hour at 37° C. After blocking, antibodies were added to the plates and incubated for 30 min at 37° C. Following incubation, 50,000 IMR90 human lung fibroblast cells or 50,000 MLG mouse fibroblast cells were then added to the wells and allowed to incubate for 2 hr at 37° C. Plates were then washed twice with PBS and the confluency of the wells was measured using the IncuCyte platform. From a high concentration single dose screen at 500 nM, 21 IgGs were identified as having >50% inhibition as shown in FIG. 1, and were carried forward to binding screens to determine relative affinities to human and mouse periostin, as shown in Table 1 below.

To determine relative affinities for recombinant human or mouse periostin, these proteins were coated on maxisorp plates overnight at 4° C. The next day, plates were blocked with casein blocking buffer for 1 hr at 37° C. Titrations of each antibody were added to the plates and allowed to bind for 1 hr at RT. Plates were washed 4× with PBST followed by incubation with an HRP conjugated anti-human Fc secondary for 30 min at RT. Plates were then washed again with 4×PBST and then developed using TMB substrate and 1M HCl. From this screen, 4 clones were selected (Table 1 marked by bold and italics) that have <1 nM binding EC50 values to both human and mouse periostin.

TABLE 1
Binding EC50 values to human and mouse periostin for the 21 IgGs
identified in the single point cell attachment assay shown in FIG l.

| EC50 | | | | | | | |
|---|---|---|---|---|---|---|---|
| (nM) | [symbol] | [symbol] | NB0629 | [symbol] | NB0640 | NB0765 | NB0776 |
| HuPOSTN | [symbol] | [symbol] | 0.09 | [symbol] | 9.30 | 1.08 | 36.60 |
| MoPOSTN | [symbol] | [symbol] | 6.08 | [symbol] | 6.41 | 2.41 | n.s. |
| EC50 | | | | | | | |
| (nM) | NB0784 | NBO791 | NB0792 | [symbol] | NB0798 | NB0800 | NB0801 |
| HuPOSTN | 4.68 | n.s. | 59.43 | [symbol] | 0.48 | n.s. | 25.16 |
| MoPOSTN | 32.76 | n.s. | 158.50 | [symbol] | 62.02 | n.s. | 22.36 |
| EC50 | | | | | | | |
| (nM) | NB0802 | NB0803 | NB0804 | NB0805 | NB0806 | NB0815 | NB0816 |
| HuPOSTN | 18.75 | 19.30 | n.s. | n.s. | n.s. | 1.05 | 1.00 |
| MoPOSTN | 25.02 | 27.44 | n.s. | n.s. | n.s. | 55.79 | n.s. |

Note that n.s. denotes that no saturation was observed in the assay.

### Example 2—Generation of NB0828 and Sequence Variants

The 4 candidates were re-tested in a dose response in the cell attachment assay to determine IC50 values. From this screen, NB0627 was identified as a particularly suitable IgG (Table 2).

TABLE 2
IC50 values for the top 4 cross-reactive
binders/blockers identified in Table 1.

| IC50 (nM) | NB0625 | NB0627 | NB0639 | NB0794 |
|---|---|---|---|---|
| HuPOSTN | 243.3 | 22.4 | 236.8 | 73.1 |

NB0627 was then converted to an effector silent IgG4PAA isotype, generating lead candidate NB0828. Sequence analysis of NB0828 identified two post translational modification liabilities in the VH region. The first, a deamidation site, is located in the CDR-H2, and the second, an oxidation site, is located in the CDR-H3. Therefore, in an attempt to remove these liabilities, several single and double mutants were generated and their binding and activity was measured. A summary of results for the IC50 and EC50 values for NB0828 and its variants are listed in Table 3.

TABLE 3
Binding and functional data summary
of NB0828 and NB0828 variants.

| | POSTN | | NB1003 | NB1010 | NB1011 |
|---|---|---|---|---|---|
| Assay | Form | NB0828 | (N55S) | (N55Q) | (N55T) |
| CAA IC50 | Human | 24.34 | 10.75 | 7.97 | 15.03 |
| (nM) | Mouse | 25.97 | 14.91 | 19.60 | 22.31 |
| Binding | Human | 0.12 | 0.08 | 0.08 | 0.07 |
| EC50 (nM) | Mouse | 0.15 | 0.08 | 0.10 | 0.07 |

| | POSTN | NB1015 | NB1012 | NB1014 |
|---|---|---|---|---|
| Assay | Form | (N55D) | (N55S_M100I) | (N55T_M100V) |
| CAA IC50 | Human | 13.63 | n.s. | n.s. |
| (nM) | Mouse | 20.91 | 42.34 | 65.08 |
| Binding | Human | 0.12 | 2.28 | 9.47 |
| EC50 (nM) | Mouse | 0.18 | 4.42 | 19.27 |

Note that n.s. denotes that no saturation was observed in the assay.

### Example 3—In Vivo Efficacy of NB0828 in Mouse Bladder MB49 and Colon CT26 Tumor Models

The efficacy of NB0828 was tested in two separate tumor models, the bladder MB49 and colon CT26 tumor models. Briefly, 250,000 MB49 cells were injected intradermally into the flank of female C57BL/6 mice, or 50,000 CT26 cells injected intradermally in the flank of female Balb/c mice. 3 days following tumor implantation, mice were treated intraperitoneal with either NB0828 (50 mg/kg, 3QW) or Vehicle Control (PBS). Tumor volume was assessed twice weekly following caliper measurement and was calculated as (length×width)/2. Mice were euthanized when tumor size exceed 15 mm in any single direction or due to tumor ulceration as a humane endpoint.

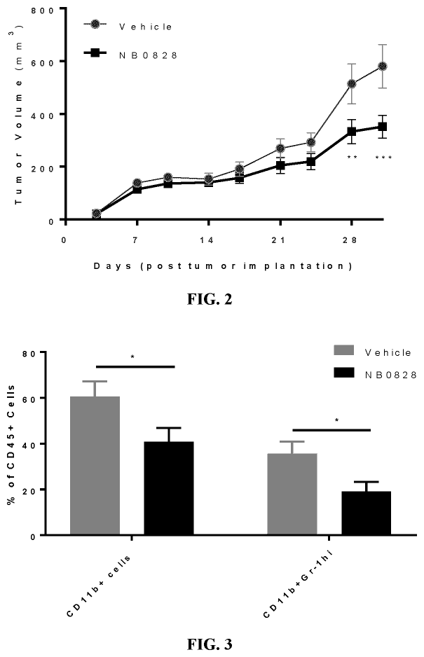

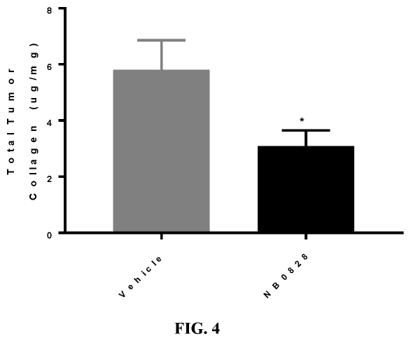

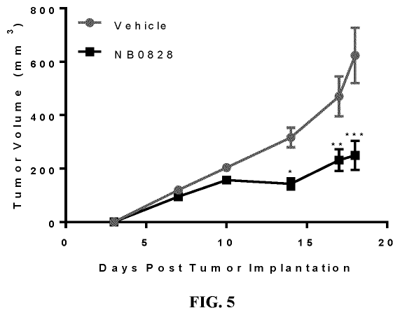

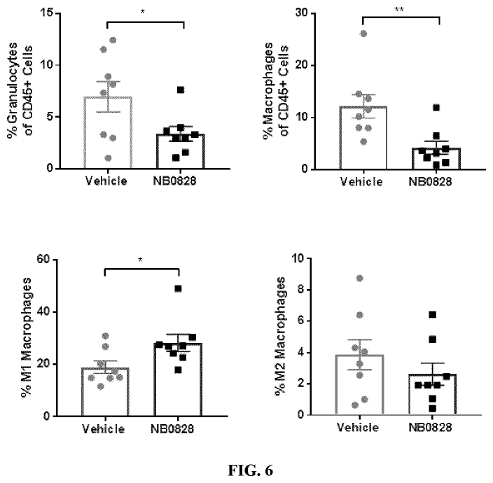

As shown in FIG. 2 and FIG. 5, NB0828 had an effect in reducing tumor growth in both models. In the MB49 model this reduction in tumor growth was associated with a lower % of intratumoral granulocytic myeloid cells as shown in FIG. 3, and a lower collagen content, as shown in FIG. 4. As with the MB49 model, the CT26 model showed a reduction in granulocytic myeloid cells. In addition, NB0828 reduced the frequency of tumor infiltrating macrophages, and the macrophages that were present were skewed towards an M1 phenotype as a result of NB0828 treatment, as shown in FIG. 6. In the CT26 mouse model, NB0828 treatment was also associated with a higher amount of tumor infiltrating CD8+ and CD4+ T cells, and a significantly higher secretion of interferon gamma in tumor infiltrating T cells as shown in FIG. 7.

### Immunophenotyping

MB49 or CT26 tumor-bearing mice were treated with NB0828 or Vehicle Control beginning on day 3 as described. For the data shown, immunophenotyping was conducted on day 20 and day 18 post tumor implantation for MB49 and CT26, respectively. Tumors were excised, skin removed, and mechanically disrupted using a scalpel blade prior to being enzymatically digested using the Miltenyi mouse tumor dissociation enzyme mix (Miltenyi Biotec, CAT #130-110-187). Digested samples were passed through a 40 μm strainer, washed in RPMI, followed by a second wash in RPMI+10% FBS. Cells were then resuspended for counting and a maximum of 2×10⁶ leukocytes per sample was plated and stained for analysis by flow cytometry. For evaluation of CD8+ tumor infiltrating lymphocyte function in the CT26 model, digested single cell suspensions from tumors were stimulated with AH1 peptide [H2-L^{d} restricted gp70 (423-431) MuLV epitope, the immunodominant CD8+ T cell epitope expressed by CT26 cells] in the presence of anti-CD28 and Brefeldin A for 5 hrs at 37° C. Following stimulation, cells were stained to detect production of IFN-γ by CD8+ T cells using flow cytometry by standard surface/intracellular staining methods. The flow staining panels used to assess cell populations shown are included below in Table 4. A viability stain (Thermo Fisher, Live/Dead Fixable Violet Stain) was used to allow interrogation of only live cell events and the pan leukocyte marker CD45 was included to allow normalization of populations within the immune compartment. Immune populations of interest were defined phenotypically/functionally as follows: Total myeloid cells (CD45+ CD11b+), granulocytes (CD45+ CD11b+ Gr-1 hi or CD45+ CD11b+ Ly6G+ Ly6C lo), Macrophages (CD45+ CD11b+ Ly6G− Ly6C lo/neg F4/80+), M1 Macrophages (MHC II+ CD206−), M2 Macrophages (MHC II− CD206+), CD8+ TILs (CD45+ CD11b− CD3+ CD90.2+ CD8+), CD4+ TILs (CD45+ CD11b− CD3+ CD90.2+ CD4+), IFN-γ+ CD8+ TILs (CD45+ CD11b− CD3+ CD8+ IFN-γ+). The Median Fluorescent Intensity (MFI) was used for determination of IFN-γ staining intensity from IFN-γ+ CD8+ TIL.

TABLE 4
Antibody cocktails used to assess immune cell phenotype/function in MB49 and CT26 tumors
MB49 Staining Panel

| | | | | |
|---|---|---|---|---|
| MHC II - AF488 | iNOS-PE | CD11b-PerCP-Cy5.5 | PD-1-PE-Cy7 | Arginase-1-APC |
| Gr-1-AF700 | CD45-APC-Fire-750 | Live/Dead Violet | F4/80 BV510 | CD206 BV650 |

CT26 Staining Panel - TIL Analysis

| | | | | |
|---|---|---|---|---|
| CD3-AF488 | PDGFR-α-PE | CD8a-PerCP-Cy5.5 | PD-1-PE-Cy7 | AH-1 Tetramer-APC |
| CD90.2-AF700 | CD45-APC-Fire-750 | Live/Dead Violet | CD4-BV510 | CD11b-BV650 |

CT26 Staining Panel - TIL Function

| | | | | |
|---|---|---|---|---|
| CD3-AF488 | IFN-γ-PE | CD8a-PerCP-Cy5.5 | PD-1-PE-Cy7 | IL-2-AF647 |
| TNF-AF700 | CD45-APC-Fire-750 | Live/Dead Violet | CD4-BV510 | CD11b-BV650 |

CT26 Staining Panel - Myeloid Cells

| | | | | |
|---|---|---|---|---|
| MHC II - AF488 | PD-L1-PE | CD11b-PerCP-Cy5.5 | Ly6C-PE-Cy7 | F4/80-AF647 |
| Ly6G-AF700 | CD45-APC-Fire-750 | Live/Dead Violet | CD40 BV510 | CD206 BV650 |

### Collagen Content

Total collagen content of tumors was assessed by quantification of hydroxyproline using the QuickZyme® total collagen assay (QuickZyme Biosciences, Leiden, The Netherlands, catalog number: QZBtotcol1). For sample preparation, MB49 tumors were excised from tumor-bearing mice when tumors had reached endpoint and were snap frozen in liquid nitrogen and stored at −80° C. prior to analysis. Tumor material was weighed and resuspended in 6M HCl at 200 mg tumor/ml HCl, vortexed and incubated at 95° C. for 20 hrs. Tubes were cooled, centrifuged at 13,000 RPM for 10 minutes, and supernatant was collected. Supernatants were diluted in Milli Q water, followed by 4M HCl according to the manufacturer's recommended protocol and were plated in technical duplicates for detection of hydroxyproline using the supplied buffers and detection reagents. OD570 nm values were measured and compared to a standard curve generated using supplied collagen to calculate the amount of collagen in each sample. Calculated total collagen (μg) for each sample was divided by the total mass of tumor input (mg) to normalize the data across tumor samples.

### Example 4—In Vivo Efficacy of NB0828 in the Mouse Colon MC38 Tumor Model

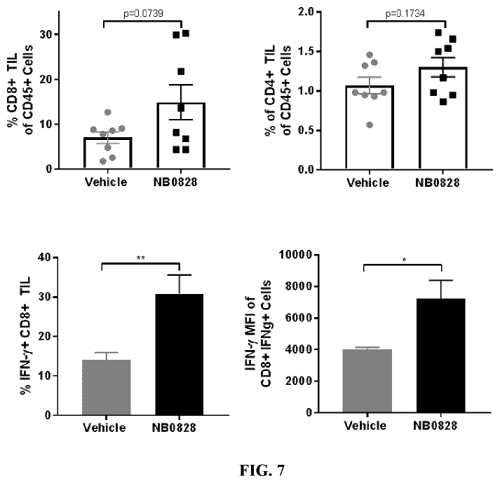

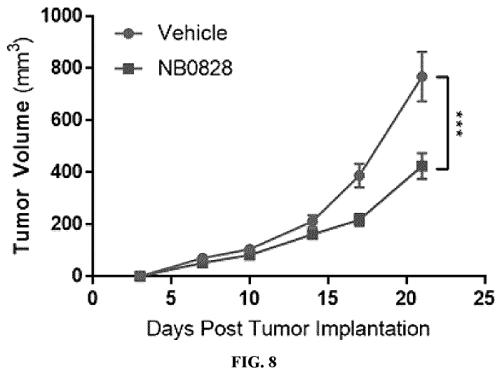

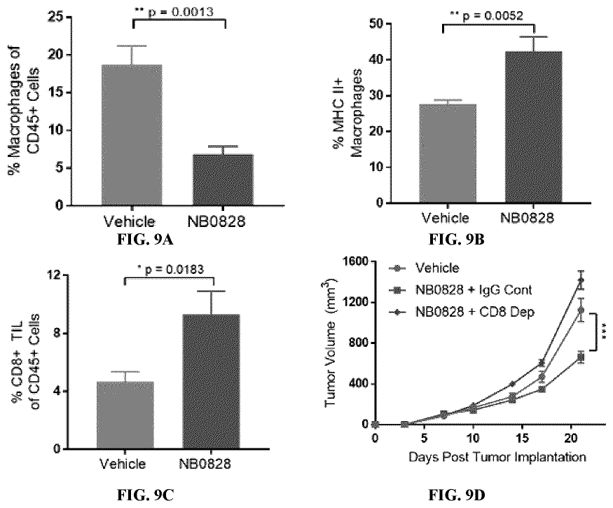

NB0828 was tested for its efficacy in reducing tumor growth in the mouse colon MC38 tumor model with the results shown in FIG. 8. NB0828 was also effective in altering the tumor microenvironment to increase CD8+ T cells, decrease the frequency of tumor-associated macrophages (TAMs) and increase the ratio of pro-inflammatory type 1 macrophages, as shown in FIGS. 9A and 9C. These changes were responsible for NB0828 efficacy in this model as depletion of CD8+ T cells during NB0828 treatment reversed the beneficial effect of NB0828, as shown in 9D. Overall, this data indicates that NB0828 allows for the increase of CD8+ T cells to tumor sites, decreases TAMs and increases the pro-inflammatory M1 macrophage phenotype in infiltrating tumors.

### Example 5—In Vivo Efficacy of NB0828 and Anti-PD-1 Antibody in the Mouse Colon MC38 Tumor Model

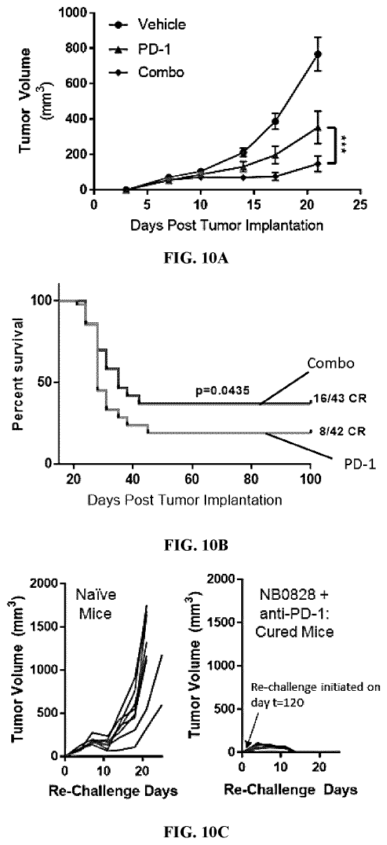

NB0828 was tested for its efficacy in reducing tumor growth in the mouse colon MC38 tumor model when administered in combination with a PD-1 function-blocking antibody. FIGS. 10A to C show that compared to anti-PD-1 alone, the combination of anti-PD-1 and NB0828 further reduced tumor growth (10A) and increased overall survival (10B complete response (CR) of 16/43 compared to 8/42)). Surviving mice that exhibited a complete response to NB0828 and anti-PD-1 combination treatment were protected from rechallenge with 10× of MC38 tumor cells indicating that surviving mice had gained immunological memory to the tumor.

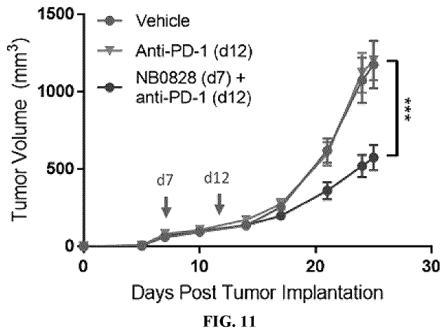

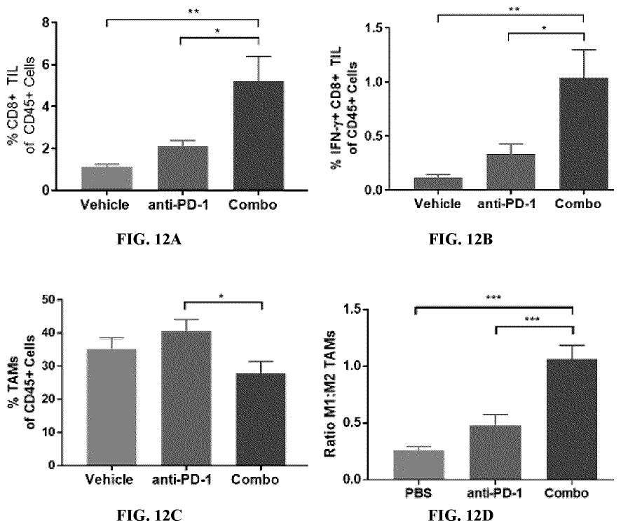

When mice with established MC38 tumors that were resistant to anti-PD-1 treatment alone were treated with the combination of anti-PD-1 and NB0828, resistance was reversed, as shown in FIG. 11. As shown in FIG. 12, combination of NB0828 and anti-PD-1 increased the infiltration and function of CD8+ T cells (FIGS. 12A and 12B), reduced TAM accumulation (FIG. 12C) and increased the ratio of pro-inflammatory phenotype of the remaining macrophages within the tumor (FIG. 12D). These modifications in the tumor microenvironment are consistent with NB0828 overcoming anti-PD-1 resistance by improving the CD8 T cell response and reducing immune-suppressive macrophages.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequence listings provided herein

| SEQ | | |
|---|---|---|
| ID | | |
| NO: | Sequence | Origin |
| 13 | QVQLVQSGAEVKKPGASVKVSCKASGYTFTSY | |
| | GISWVRQAPGQGLEWMGWISAYNGNTNYAQKL | |
| | QGRVTMTTDTSTSTAYMELRSLRSDDTAVYYC | |
| | ARDMLVVPFDYWGQGTLVTVSS | |
| 14 | QSVLTQSSSASGTPGQTVTVSCSGSSSDIGSN | |
| | RVNWYQQLPGTAPKLLIYSNDQRPSGVPDRFS | |
| | GSKSGTSASLAISGLQSADEADYYCAAWDDSL | |
| | STYVFGSGTKVTVL | |
| 19 | EVMLVESGGGLVQPGGSLRLSCTASGFTFSKS | Ezabenlimab |
| | AMSWVRQAPGKGLEWVAYISGGGGDTYYSSSV | (CAS # |
| | KGRFTISRDNAKNSLYLQMNSLRAEDTAVYYC | 2249882- |
| | ARHSNVNYYAMDYWGQGTLVTVSSASTKGPSV | 54-8) |
| | FPLAPCSRSTSESTAALGCLVKDYFPEPVTVS | HC |
| | WNSGALTSGVHTFPAVLQSSGLYSLSSVVTVP | |
| | SSSLGTKTYTCNVDHKPSNTKVDKRVESKYGP | |
| | PCPPCPAPEFLGGPSVFLFPPKPKDTLMISRT | |
| | PEVTCVVVDVSQEDPEVQFNWYVDGVEVHNAK | |
| | TKPREEQFNSTYRVVSVLTVLHQDWLNGKEYK | |
| | CKVSNKGLPSSIEKTISKAKGQPREPQVYTLP | |
| | PSQEEMTKNQVSLTCLVKGFYPSDIAVEWESN | |
| | GQPENNYKTTPPVLDSDGSFFLYSRLTVDKSR | |
| | WQEGNVFSCSVMHEALHNHYTQKSLSLSLG | |
| 20 | EIVLTQSPATLSLSPGERATMSCRASENIDVS | Ezabenlimab |
| | GISFMNWYQQKPGQAPKLLIYVASNQGSGIPA | (CAS # |
| | RFSGSGSGTDFTLTISRLEPEDFAVYYCQQSK | 2249882- |
| | EVPWTFGQGTKLEIKRTVAAPSVFIFPPSDEQ | 54-8) |
| | LKSGTASVVCLLNNFYPREAKVQWKVDNALQS | LC |
| | GNSQESVTEQDSKDSTYSLSSTLTLSKADYEK | |
| | HKVYACEVTHQGLSSPVTKSFNRGEC | |

1. A method of treating an individual afflicted with a cancer, the method comprising administering to the individual (a) a PD-1 axis inhibitor; and (b) an inhibitor of periostin.

2. The method of claim 1, wherein the inhibitor of periostin comprises an antibody or antigen binding fragment thereof that binds periostin.

3. The method of claim 2, wherein the inhibitor of periostin comprises an antibody or antigen binding fragment thereof that binds periostin, wherein the antibody or antigen binding fragment that binds periostin thereof comprises:
a) an immunoglobulin heavy chain CDR1 (CDR-H1) comprising the amino acid sequence set forth in SEQ ID NO: 1 (GYTFTSYG);
b) an immunoglobulin heavy chain CDR2 (CDR-H2) comprising an amino acid sequence set forth in any one of SEQ ID NOs: 2 (ISAYNGNT), 3 (ISAYSGNT), 4 (ISAYQGNT), 5 (ISAYTGNT), or 6 (ISAYDGNT);
c) an immunoglobulin heavy chain CDR3 (CDR-H3) comprising an amino acid sequence set forth in any one of SEQ ID NOs: 7 (DILVVPFDY), 8 (DVLVVPFDY), or 9 (DMLVVPFDY);
d) an immunoglobulin light chain CDR1 (CDR-L1) comprising the amino acid sequence set forth in SEQ ID NO: 10 (SSDIGSNR);
e) an immunoglobulin light chain CDR2 (CDR-L2) amino comprising the amino acid sequence set forth in SEQ ID NO: 11 (SND); and
f) an immunoglobulin light chain CDR3 (CDR-L3) comprising the amino acid sequence set forth in SEQ ID NO: 12 (AAWDDSLSTYV).

4. The method of claim 3, wherein the recombinant antibody or antigen binding fragment thereof that binds periostin is chimeric or humanized.

5. The method of claim 3, wherein the recombinant antibody or antigen binding fragment thereof that binds periostin is an IgG antibody.

6. The method of claim 3, wherein the recombinant antibody or antigen binding fragment thereof that binds periostin is a Fab, F(ab)₂, a single-domain antibody, or a single chain variable fragment (scFv).

7. The method of claim 3, wherein the antibody or antigen binding fragment that binds periostin thereof comprises immunoglobulin heavy chain variable region and an immunoglobulin light chain variable region:
a) wherein the immunoglobulin heavy chain variable region comprises an amino acid sequence at least about 90%, 95%, 97%, 99%, or 100% identical to that set forth in SEQ ID NO: 13; and
b) wherein the immunoglobulin light chain variable region comprises an amino acid sequence at least about 90%, 95%, 97%, 99%, or 100% identical to that set forth in SEQ ID NO: 14, wherein asparagine number 55 of SEQ ID NO: 13 is asparagine, serine, glutamine, threonine, or aspartic acid, and wherein methionine number 100 of SEQ ID NO: 13 is methionine, isoleucine, or valine.

8. A method of treating an individual afflicted with a cancer, the method comprising administering to the individual (a) a PD-1 axis inhibitor; and (b) an inhibitor of periostin.

9. The method of claim 8, wherein the inhibitor of periostin comprises an antibody or antigen binding fragment thereof that binds periostin.

10. The method of claim 9, wherein the inhibitor of periostin comprises an antibody or antigen binding fragment thereof that binds periostin, the antibody or antigen binding fragment thereof that binds periostin comprising an immunoglobulin heavy chain variable region and an immunoglobulin light chain variable region:
a) wherein the immunoglobulin heavy chain variable region comprises an amino acid sequence at least about 90%, 95%, 97%, 99%, or 100% identical to that set forth in SEQ ID NO: 13; and
b) wherein the immunoglobulin light chain variable region comprises an amino acid sequence at least about 90%, 95%, 97%, 99%, or 100% identical to that set forth in SEQ ID NO: 14, wherein asparagine number 55 of SEQ ID NO 13: is asparagine, serine, glutamine, threonine, or aspartic acid, and wherein methionine number 100 of SEQ ID NO: 13 is methionine, isoleucine, or valine.

11. The method of claim 10, wherein the antibody or antigen binding fragment thereof that binds periostin is chimeric or humanized.

12. The method of claim 10, wherein the antibody or antigen binding fragment thereof that binds periostin is an IgG antibody.

13. The method of claim 10 or 11, wherein the antibody or antigen binding fragment thereof that binds periostin is a Fab, F(ab)₂, a single-domain antibody, or a single chain variable fragment (scFv).

14. The method of claim 3, wherein the antibody or antigen binding fragment thereof that binds periostin has an IC50 of less than about 50 nanomolar in a cell adhesion assay performed with human lung fibroblast cells and/or mouse fibroblast cells.

15. The method of claim 1, wherein the PD-1 axis inhibitor is an inhibitor of PD-1, PDL-1, or PDL-2 signaling is an antibody or fragment thereof that binds to PD-1.

16. The method of claim 15, wherein the PD-1 axis inhibitor is an antibody or fragment thereof that binds to PD-1.

17. The method of claim 15, wherein the antibody or fragment thereof that binds to PD-1 comprises a heavy chain comprising the amino acid sequence of SEQ ID NO.:19 and a light chain comprising the amino acid sequence of SEQ ID NO.:20.

18. The method of claim 15, wherein the antibody or fragment thereof that binds to PD-1 comprises pembrolizumab, nivolumab, pidilizumab, tislelizumab, spartalizumab, AMP-514 (MEDI0680), or ezabenlimab, or a PD-1 binding fragment thereof.

19. The method of claim 15, wherein the PD-1 axis inhibitor is an antibody that specifically binds PDL-1 or PDL-2.

20. The method of claim 9, wherein the antibody that specifically binds PDL-1 or PDL-2 comprises durvalumab, atezolizumab, avelumab, BMS-936559 (MDX-1105), AMP-224, cemiplimab (REGN2810), toripalimab (JS001-PD-1), camrelizumab (SHR-1210), dostarlimab (TSR-042), cetrelimab (JNJ-63723283), or FAZ053, or a PDL-1 or PDL-2 binding fragment thereof.

21. The method of claim 15, wherein the inhibitor of PD-1, PDL-1, or PDL-2 signaling comprises an Fc-Fusion protein that binds PD-1, PDL-1, or PDL-2.

22. The method of claim 21, wherein the Fc-Fusion protein comprises AMP-224 or a PD-1 binding fragment thereof.

23. The method of claim 8, wherein the inhibitor of PD-1, PDL-1, or PDL-2 signaling comprises a small molecule inhibitor of PD-1, PDL-1, or PDL-2.

24. The method of claim 23, wherein the small molecule inhibitor of signaling through PD-1, PDL-1, or PDL-2 comprises on or more of: N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo[b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-α-glutamyl-L-seryl-L-phenylalanyl)-L-lysyl-L-phenylalanyl-L-arginyl-L-valyl-L-threonyl-L-glutaminyl-L-leucyl-L-alanyl-L-prolyl-L-lysyl-L- alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L-tryptophyl-N-methyl-L-norleucyl-N-methyl-L- norleucyl-L-arginyl-L-cysteinyl-, cyclic (1→14)-thioether; or a derivative or analog thereof.

25. The method of claim 1, wherein the individual has developed progressive disease after treatment with a checkpoint inhibitor as a monotherapy.

26. The method of claim 25, wherein the checkpoint inhibitor comprises a PD-1 access inhibitor.

27. The method of claim 1, wherein the PD-1 axis inhibitor and the inhibitor of periostin are administered separately.

28. The method of claim 1, wherein the PD-1 axis inhibitor and the inhibitor of periostin are administered on the same day.

29. The method of claim 1, wherein the PD-1 axis inhibitor and the inhibitor of periostin are administered on different days.

30. The method of claim 1, wherein the cancer comprises glioblastoma, pancreatic cancer, breast cancer, bladder cancer, kidney cancer, head and neck cancer, ovarian cancer, colon cancer, cervical cancer, prostate cancer, or lung cancer.

31.-53. (canceled)


SEQUENCE LISTING (22 sequences)

SEQ ID NO: 1 - protein, 8 aa, Artificial Sequence
  Description of Artificial Sequence Synthetic peptide
GYTFTSYG

SEQ ID NO: 2 - protein, 8 aa, Artificial Sequence
  Description of Artificial Sequence Synthetic peptide
ISAYNGNT

SEQ ID NO: 3 - protein, 8 aa, Artificial Sequence
  Description of Artificial Sequence Synthetic peptide
ISAYSGNT

SEQ ID NO: 4 - protein, 8 aa, Artificial Sequence
  Description of Artificial Sequence Synthetic peptide
ISAYQGNT

SEQ ID NO: 5 - protein, 8 aa, Artificial Sequence
  Description of Artificial Sequence Synthetic peptide
ISAYTGNT

SEQ ID NO: 6 - protein, 8 aa, Artificial Sequence
  Description of Artificial Sequence Synthetic peptide
ISAYDGNT

SEQ ID NO: 7 - protein, 9 aa, Artificial Sequence
  Description of Artificial Sequence Synthetic peptide
DILVVPFDY

SEQ ID NO: 8 - protein, 9 aa, Artificial Sequence
  Description of Artificial Sequence Synthetic peptide
DVLVVPFDY

SEQ ID NO: 9 - protein, 9 aa, Artificial Sequence
  Description of Artificial Sequence Synthetic peptide
DMLVVPFDY

SEQ ID NO: 10 - protein, 8 aa, Artificial Sequence
  Description of Artificial Sequence Synthetic peptide
SSDIGSNR

SEQ ID NO: 11 - protein, 3 aa, Artificial Sequence
  Description of Artificial Sequence Synthetic peptide
SND

SEQ ID NO: 12 - protein, 11 aa, Artificial Sequence
  Description of Artificial Sequence Synthetic peptide
AAWDDSLSTYV

SEQ ID NO: 13 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDMLVVPFDYWGQGTLVTVSS

SEQ ID NO: 14 - protein, 110 aa, Artificial Sequence
  Description of Artificial Sequence Synthetic polypeptide
QSVLTQSSSASGTPGQTVTVSCSGSSSDIGSNRVNWYQQLPGTAPKLLIYSNDQRPSGVPDRFSGSKSGTSASLAISGLQSADEADYYCAAWDDSLSTYVFGSGTKVTVL

SEQ ID NO: 15 - protein, 836 aa, Unknown
  Description of Unknown Periostin sequence
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 16 - protein, 8 aa, Artificial Sequence
  Description of Artificial Sequence Synthetic peptide
  MOD_RES (5)..(5): Any amino acid
ISAYXGNT

SEQ ID NO: 17 - protein, 9 aa, Artificial Sequence
  Description of Artificial Sequence Synthetic peptide
  MOD_RES (2)..(2): Any amino acid
DXLVVPFDY

SEQ ID NO: 18 - protein, 6 aa, Artificial Sequence
  Description of Artificial Sequence Synthetic 6xHis tag
HHHHHH

SEQ ID NO: 19 - protein, 446 aa, Artificial Sequence
  Description of Artificial Sequence Synthetic polypeptide
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 218 aa, Artificial Sequence
  Description of Artificial Sequence Synthetic polypeptide
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 21 aa, Artificial Sequence
  Description of Artificial Sequence Synthetic peptide
  MOD_RES (5)..(5): Alpha-Glu
SNTSESFKFRVTQLAPKAQIK

SEQ ID NO: 22 - protein, 14 aa, Artificial Sequence
  Description of Artificial Sequence Synthetic peptide
  MOD_RES (2)..(2): N-Methyl-Ala
  MOD_RES (7)..(7): N-Methyl-Gly
  MOD_RES (11)..(11): N-Methyl-Norleucine
  MOD_RES (12)..(12): N-Methyl-Norleucine
FANPHLGWSWLLRC